(19) United States
(12) Patent Application Publication
(10) Pub. No.: US 2015/0210746 A1
(43) Pub. Date: Jul. 30, 2015

(54) METHOD FOR EXPRESSION, EXTRACTION AND PURIFICATION OF SOLUBLE RECOMBINANT PROTEIN

(71) Applicant: KOREA RESEARCH INSTITUTE OF STANDARDS AND SCIENCE, Daejeon (KR)

(72) Inventors: Jun-Hyuk Choi, Daejeon (KR); Sook-Kyung Kim, Daejeon (KR); Minji Kim, Daegu (KR)

(73) Assignee: KOREA RESEARCH INSTITUTE OF STANDARDS AND SCIENCE, Daejeon (KR)

(21) Appl. No.: 14/657,228

(22) Filed: Mar. 13, 2015

Related U.S. Application Data
(63) Continuation-in-part of application No. PCT/KR2013/008452, filed on Sep. 17, 2013.

(30) Foreign Application Priority Data
Sep. 19, 2012 (KR) 10-2012-0104252

(51) Int. Cl. C07K 14/61; B01D 15/42; B01D 15/34; B01D 15/38; B01D 15/36

(52) U.S. Cl. CPC C07K 14/61; B01D 15/3823; B01D 15/363; B01D 15/34; B01D 15/426

(57) ABSTRACT

The present invention relates to a method for expressing, extracting and refining recombinant human growth hormone (hGH). More particularly, the present invention relates to a method for the intracellular expression of a target protein with minimizing the formation of insoluble inclusion bodies during the mass production of the protein in Escherichia coli system. The present invention also relates to a method for extracting and refining the target protein, particularly human growth hormone, with maximizing its solubility during the extraction and with providing a high yield but without losing the biological activity of the target protein.

### CROSS REFERENCE TO RELATED APPLICATION

This is a Continuation-in-part of International Application No. PCT/KR2013/008452, filed Sep. 17, 2013, which in turn claims the benefit of Korean Patent Application No. 10-2012-0104252, filed Sep. 19, 2012. The Korean application is incorporated herein in its entirety.

### BACKGROUND OF THE INVENTION

1. Field of the Invention

The present invention relates to a method for expressing, extracting and refining a soluble target protein. More particularly, the present invention relates to a method for the intracellular expression of a target protein with minimizing the formation of insoluble inclusion bodies during the mass production of the protein in Escherichia coli system, and a method for extracting and refining the target protein, particularly human growth hormone, with maximizing its solubility during the extraction and with providing a high yield but without losing the biological activity of the target protein.

2. Description of the Related Art

As recombinant DNA related techniques and protein purification methods advance, an industrial process has been set up for the mass production of a recombinant protein. In particular, a simple and economic purification and production process is a great advantage for the industrialization. The production, separation, and purification methods differ from the characteristics of each recombinant protein. However, there are general methods largely carried out, which are as follows; (1) A specific affinity tag is conjugated to a target protein, followed by expressing thereof. Then, the protein is purified by using an affinity resin binding specifically to the tag. This method, however, often requires the step of eliminating the affinity tag, for which the processes of tag cutting using a specific enzyme and of additional purification are also required. (2) A target protein is expressed without a tag. Then, the protein is purified by using a selected chromatography stepwise according to the characteristics of the protein. In this method, the optimization of the conditions for the expression, separation, and purification has to be coordinated in advance to maximize the yield of the target protein.

Human growth hormone (hGH) is composed of 191 amino acid residues, which is a single chain polypeptide synthesized in the pituitary gland. It is well known that hGH, one of the most important human hormones, has various biological functions in relation to cell growth and metabolism (Annu Rev Physiol 47, 1985, 483-499). The endogenously expressed hGH is not glycosylated. So, the method for the mass production of hGH in the form of a recombinant protein using an expression system of prokaryotes is widely performed.

However, when hGH is massively expressed in E. coli, the insoluble protein coagulate, so called insoluble inclusion body, is generated, which is often observed during the protein expression in prokaryotes (Gene 165, 1995, 303-306). Therefore, the steps of solubilization and refolding are necessary to dissolve the hGH inclusion bodies with a chemical material before the purification process by chromatography. The total protein yield ratio is significantly affected by the efficiency of the pre-purification process such as the above. In general, in order to increase the solubility of the coagulated proteins, high concentration denaturants such as Urea or GnHC1 are used and then the used denaturant is eliminated for the next refolding process of the protein. However, the method for recovering the protein from such insoluble inclusion bodies often causes damage on the biological activity of the protein. Besides, the cost for purification is still high and the time is still consumed a lot (Biotechnology(NY) 11; 1993, 349-57).

To overcome the above problems, new methods and techniques have been developed to increase the solubility of hGH and for efficient refolding and yield as well with keeping the biological activity of the protein (Biotechnol Prog 14, 1998, 722-728; J Bioxei Bioeng 99, 2005, 303-310; Biosci Biotechnol Biochem 72, 2008, 2675-2680; J Biol Chem 276, 2001, 46856-46863). To prevent the generation of such inclusion body, a method for inducing the secretion of hGH in cytoplasm of bacteria has been developed (FEBS Lett 204, 1986, 145-150). However, it is still difficult to establish a method for dissolving an insoluble recombinant protein produced during the mass-expression thereof in prokaryotic system and a method for separating and refining the recombinant protein with keeping the biological activity with high yield.

In the course of study to develop a method to dissolve an insoluble recombinant protein efficiently and a method for separating and purifying the recombinant protein with high yield, the present inventors were able to succeed in optimizing the expression, extraction, and purification of a target protein by using E. coli expression system. As a result, the generation of insoluble inclusion bodies was minimized and the solubility of the recombinant protein, particularly human growth hormone, was maximized, suggesting that the high purity recombinant protein was obtained with high yield. Therefore, the present inventors completed this invention by confirming that the method of the invention could be effectively used for the mass-production of a target protein having excellent biological activity.

### PRIOR PUBLICATION

### Patent Documents

Korean Patent Publication No. 1997-0006498
Korean Patent Publication No. 1999-0016368
Korean Patent Publication No. 1999-0069476

### Non-Patent Documents

Annu Rev Physiol 47, 1985, 483-499
Gene 165, 1995, 303-306
Biotechnology(NY) 11, 1993, 349-57
Biotechnol Prog 14, 1998, 722-728
J Bioxei Bioeng 99, 2005, 303-310
Biosci Biotechnol Biochem 72, 2008, 2675-2680
J Biol Chem 276, 2001, 46856-46863
FEBS Lett 204, 1986, 145-150

### SUMMARY OF THE INVENTION

It is an object of the present invention to provide an intracellular expression method of a target protein, particularly hGH, in prokaryotic system with minimizing the generation of the inclusion body of hGH, and an extraction and purification method of the target protein efficient in maximizing the solubility of the target protein during the extraction process.

It is another object of the present invention to provide an efficient protein expression and extraction method which can not only give high yield but also overcome the problems of the conventional method such as low total yield of a biologically active protein, high purification cost, and time-consuming labor, and to provide an extraction and purification method optimized for the mass-production of a soluble target protein with maintaining its biological activity.

To achieve the above objects, the present invention provides a method for producing a soluble target protein comprising the following steps:

(1) performing primary culture of E. coli to express a target protein therein;

(2) quick-freezing the E. coli primary culture fluid at 0˜10° C., which stands as it is for 30˜180 minutes;

(3) adding an inducer to the E. coli culture fluid to induce the expression of the target protein; and

(4) culturing the E. coli culture fluid added with the said inducer at 15˜25° C. for 8˜18 hours.

The present invention also provides a method for extracting and purifying a soluble target protein comprising the following steps:

(1) lysing the E. coli cells by using a lysis buffer;

(2) sonicating the E. coli cells, which are then centrifuged to recover the soluble target protein; and

(3) purifying the soluble target protein.

### Advantageous Effect

The present invention relates to a method for the intracellular expression of a recombinant protein with minimizing the formation of insoluble inclusion bodies during the mass expression of the protein in E. coli system, and an extraction method efficient in minimizing the protein coagulation observed during the collection of the recombinant protein from the E. coli cells, and an efficient purification method using chromatography to obtain a high purity recombinant protein with biological activity.

In particular, when the recombinant protein of the present invention is human growth hormone, the method for mass-expression, extraction, and purification of the invention is advantageous not only in obtaining high yield owing to the solubilization of insoluble hGH but also in obtaining a target protein displaying excellent biological activities. Therefore, the method of the present invention can be effectively used for the mass-expression, extraction, and purification of a target protein.

### BRIEF DESCRIPTION OF THE DRAWINGS

The application of the preferred embodiments of the present invention is best understood with reference to the accompanying drawings, wherein:

FIG. 1 shows a set of photographs displaying the gel illustrating the insoluble fraction (pallet: P) and the soluble fraction (soluble: S) of human growth hormone according to the induction temperature, resulted from SDS-PAGE electrophoresis, followed by coomassie blue staining. It also shows a graph illustrating the solubility of human growth hormone over the induction temperature (solubility presented as %, indicating the concentration of soluble fraction of protein with regarding the concentration of insoluble and soluble human growth hormone as 100%). The mean value±standard error (SE) is presented in the graph.

FIG. 2 is a set of photographs of gels stained with coomassie blue after 4˜12% SDS-PAGE electrophoresis, which illustrate the expression of human growth hormone at the temperature of 37° C. (A) and the expression of human growth hormone at the temperature of 16° C. (C);

U: E. coli cells in which the expression of human growth hormone was not induced;

I: E. coli cells in which the expression of human growth hormone was induced by the treatment of IPTG;

L: E. coli cells treated with a lysis buffer after confirming the expression of human growth hormone therein;

P: insoluble fraction obtained by centrifuging the E. coli cells treated with a lysis buffer after confirming the expression of human growth hormone therein;

S: soluble fraction obtained by centrifuging the E. coli cells treated with a lysis buffer after confirming the expression of human growth hormone therein; and

*: lysozyme. (B) and (D) are diagrams illustrating the relative ratios (%) of P and S in (A) and (C).

FIG. 3 is a set of a gel photograph (A) and a graph (B) illustrating the changes of the solubility of human growth hormone observed in lysis buffer supplemented with 0.1˜1% (v/v) Triton X-100 during the process of the extraction of human growth hormone expressed at 16° C. and 37° C. In the course of the extraction of human growth hormone, since there was no big change in the solubility of the human growth hormone expressed at 37° C., it was presented as a dotted line in the graph (B).

FIG. 4 is a set of a gel photograph (A) and a graph (B) illustrating the changes of the solubility of human growth hormone observed in lysis buffer supplemented with 0.1˜1% (v/v) Tween 20 during the process of the extraction of human growth hormone expressed at 16° C. In the course of the extraction of human growth hormone, since there was no big change in the solubility of the human growth hormone expressed at 37° C., it was presented as a dotted line in the graph (B).

FIG. 5 is a set of a gel photograph (A) and a graph (B) illustrating the changes of the solubility of human growth hormone observed in lysis buffer supplemented with 0.15˜1 M NaCl during the process of the extraction of human growth hormone expressed at 16° C. In the course of the extraction of human growth hormone, since there was no big change in the solubility of the human growth hormone expressed at 37° C., it was presented as a dotted line in the graph (B).

FIG. 6 is a set of a gel photograph (A) and a graph (B) illustrating the changes of the solubility of human growth hormone observed in lysis buffer supplemented with 0.15˜1 M KCl during the process of the extraction of human growth hormone expressed at 16° C. In the course of the extraction of human growth hormone, since there was no big change in the solubility of the human growth hormone expressed at 37° C., it was presented as a dotted line in the graph (B).

FIG. 7 is a set of a gel photograph (A) and a graph (B) illustrating the changes of the solubility of human growth hormone observed in lysis buffer supplemented with 5˜20 mM β-mercaptoethanol during the process of the extraction of human growth hormone expressed at 16° C. In the course of the extraction of human growth hormone, since there was no big change in the solubility of the human growth hormone expressed at 37° C., it was presented as a dotted line in the graph (B).

FIG. 8 is a set of a gel photograph (A) and a graph (B) illustrating the changes of the solubility of human growth hormone according to the volume (ml) of the lysis buffer during the process of the extraction of human growth hormone expressed at 16° C. 0.25˜2 ml of lysis buffer was added to 30 mg of pellet (insoluble protein). In the course of the extraction of human growth hormone, since there was no big change in the solubility of the human growth hormone expressed at 37° C., it was presented as a dotted line in the graph (B).

FIG. 9 is a set of a gel photograph and a graph illustrating the relative recovery rate (%) of the insoluble fraction to the soluble fraction of His-hGH primarily separated under the optimized condition for the extraction after being expressed at 16° C. for 16 hours:

optimized lysis buffer: 50 mM Tris-HCl (pH 8.0) containing 0.5 mM EDTA, 0.1% Triton X-100, 1 mg/ml lysozyme, and 1× protease inhibitor cocktail;

volume of lysis buffer: 1 ml for 30 mg E. coli cell pellet (insoluble protein);

P: insoluble protein (pellet); and

S: soluble protein.

FIG. 10 is a flow chart illustrating the purification process of His-hGH extracted from E. coli.

FIG. 11 is a set of photographs of SDS-PAGE gel displaying His-hGH obtained from each purification process:

U: E. coli cells in which the expression of human growth hormone was not induced;

I: E. coli cells in which the expression of human growth hormone was induced by the treatment of IPTG;

L: E. coli cells treated with a lysis buffer after confirming the expression of human growth hormone therein;

P: insoluble fraction obtained by centrifuging the E. coli cells treated with a lysis buffer after confirming the expression of human growth hormone therein;

S: soluble fraction obtained by centrifuging the E. coli cells treated with a lysis buffer after confirming the expression of human growth hormone therein; and Ni-NTA: after the purification with NiNTA column;

Q: after the purification of the protein already purified with NiNTA column once by using Mono Q-column;

SEC: after the purification of the protein already purified with NiNTA column and Mono Q-column by using Superdex column; and

*: lysozyme.

FIG. 12 is a set of a gel photograph and a graph illustrating the relative recovery rate (%) of the insoluble fraction to the soluble fraction of untagged hGH primarily separated under the optimized condition for the extraction after being expressed at 16° C. for 16 hours:

optimized lysis buffer: 50 mM Tris-HCl (pH 8.0) containing 0.5 mM EDTA, 0.1% Triton X-100, 1 mg/ml lysozyme, and 1× protease inhibitor cocktail.

FIG. 13 is a flow chart illustrating the purification process of untagged hGH extracted from E. coli.

FIG. 14 is a set of photographs of SDS-PAGE gel displaying untagged hGH obtained from each purification process:

U: E. coli cells in which the expression of human growth hormone was not induced;

I: E. coli cells in which the expression of human growth hormone was induced by the treatment of IPTG;

L: E. coli cells treated with a lysis buffer after confirming the expression of human growth hormone therein;

P: insoluble fraction obtained by centrifuging the E. coli cells treated with a lysis buffer after confirming the expression of human growth hormone therein;

S: soluble fraction obtained by centrifuging the E. coli cells treated with a lysis buffer after confirming the expression of human growth hormone therein; and

DEAE: after the purification with DEAE column;

Q: after the purification of the protein already purified with DEAE column once by using Mono Q-column;

SEC: after the purification of the protein already purified with DEAE column and Mono Q-column by using Superdex column; and

*: lysozyme.

FIG. 15 is a RP-HPLC chromatogram graph of the purified human growth hormone. The unit of vertical axis indicates calculated mV.

FIG. 16 is a diagram illustrating the result of analysis with human growth hormone using size exclusion chromatography (SEC) (200 kDa; blue dextran, 66 kDa: BSA, 29 kDa: carbonic anhydrase, and 12.4 kDa: ribonuclease A, These were used as molecular markers and displayed by the log size).

FIG. 17 is a diagram illustrating the result of analysis with the purified circular human growth hormone using MALDI-TCF mass spectrometry (performed in cation mode, m/z: 15˜45 kDa).

FIG. 18 is a diagram illustrating the purified human growth hormone analyzed by circular dichroism (CD). CD spectra of the control hGH, His-hGH, and untagged hGH were scanned in the range of 190˜250 nm.

FIG. 19 is a diagram illustrating the purified human growth hormone analyzed by NB2-11 cell proliferation assay. Nb2-11 cell proliferation was suppressed by serum deprivation. Then, the cells were treated with control hGH (□), His-hGH (▴), untagged hGH (◯), or BSA (♦), followed by culture for 48 hours. The cell number was counted by the manner described in Example 5. The counting was performed in triplicate and the average was calculated and presented as mean value±standard error in the graph.

### DESCRIPTION OF THE PREFERRED EMBODIMENTS

Hereinafter, the present invention is described in detail.

The present invention provides a method for producing a soluble target protein comprising the following steps:

(1) performing primary culture of E. coli transfected with an expression vector encoding a target protein;

(2) quick-freezing the E. coli primary culture fluid at 0˜10° C., which stands as it is for 30˜180 minutes;

(3) adding an inducer to the E. coli culture fluid to induce the expression of the target protein; and

(4) culturing the E. coli culture fluid added with the said inducer at 15˜25° C. for 8˜18 hours.

In step (1), the target protein can be any protein generally used by those in the art, but human growth hormone (hGH) is preferred, but not always limited thereto. In step (1), the E. coli is preferably E. coli BL21, and the primary culture is preferably performed at 37° C. until OD₆₀₀ reaches 0.6, but not always limited thereto. The culture medium is preferably a fresh LB (Luria-Bretanu) medium supplemented with 50 mg/ml of kanamycin, 10 g/l of Bacto Tryptone, 5 g/l of yeast extract, and 10 g/l of NaCl, but not always limited thereto.

In step (2), the quick-freezing and standing preferably indicates the quick-freezing at 0˜10° C. and standing as it is for 30˜180 minutes, and more preferably indicates quick-freezing at 2˜4° C. and standing as it is for 40˜60 minutes, but not always limited thereto. Such quick-freezing and standing is to suppress the cell growth quickly in order to maximize the expression of the recombinant protein induced by a protein expression inducer added thereto later on and at the same time to minimize the generation of insoluble inclusion bodies.

In step (3), β-D-1-thiogalactopyranoside (IPTG), as an inducer, that strongly induces the enzyme synthesis of E. coli lactose operon, is treated to the E. coli culture fluid stored in a refrigerator at the concentration of 0.1˜1 mM. Then, the protein expression is preferably induced at 15˜25° C. for 8˜18 hours, and more preferably induced at 16˜20° C. for 12˜16 hours. That is, the human growth hormone expression is induced by the added inducer and this way of inducing the expression under the said condition is the best way to minimize the generation of insoluble inclusion bodies.

The present invention also provides a method for extracting and purifying a soluble target protein comprising the following steps:

(1) performing primary culture of E. coli transfected with an expression vector encoding a target protein;

(2) quick-freezing the E. coli primary culture fluid at 0˜10° C., which stands as it is for 30˜180 minutes;

(3) adding an inducer to the E. coli culture fluid to induce the expression of the target protein;

(4) culturing the E. coli culture fluid added with the said inducer at 15˜25° C. for 8˜18 hours.
(5) lysing the E. coli cells of step (4) by using a lysis buffer;

(6) sonicating the E. coli cells, which are then centrifuged to recover the soluble target protein; and
(7) purifying the soluble target protein.

The soluble target protein herein is preferably the biologically active protein in which the insoluble protein coagulate so called the inclusion body is not formed.

In the above method, the lysis buffer is preferably Tris-HCl (pH 8.0) containing 0.5 mM EDTA, 1 mg/ml lysozyme, 1× protease inhibitor cocktail and a non-ionic detergent, but not always limited thereto.

The said non-ionic detergent is preferably Triton X-100 or Tween 20. The concentration of the non-ionic detergent is preferably 0.01˜2% (v/v), and more preferably 0.1˜1% (v/v).

The amount of the said lysis buffer is preferably 0.25˜2 ml for 30 mg of the E. coli cells (pellet) expressing insoluble human growth hormone, and more preferably 1 to dissolve the human growth hormone.

In the meantime, when NaCl or KCl was used in the lysis buffer to dissolve the insoluble human growth hormone, this salt was not effective in dissolving the protein and rather reduce the solubility. So, the lysis buffer of the present invention characteristically does not include such salt. It was also confirmed that the solubility before and after the addition of β-mercaptoethanol was not changed and the concentration of β-mercaptoethanol did not affect the solubility, either. So, β-mercaptoethanol is not necessarily included in the lysis buffer herein.

The concentrations of Triton X-100 and Tween 20 are preferably 0.1˜1% (v/v). If the concentration is less than 0.1% (v/v), the solubilization effect is very weak. On the other hand, if the concentration is more than 1% (v/v), the solubilization effect is not increased any more, suggesting that excessive amount of detergent is inefficient.

In the optimum condition of the present invention, hGH, the recombinant protein massively expressed in E. coli is characteristically extracted 90˜95% in the soluble form.

In the method for extracting and purifying a soluble target protein of the present invention, the soluble target protein is characteristically purified by one or more methods selected from the group consisting of affinity chromatography, anion exchange chromatography, and gel-filter chromatography, after the protein extraction.

For example, histidine tagged human growth hormone (His-hGH) was firstly purified by affinity chromatography using Ni-NTA column and then secondarily purified by anion exchange chromatography using Mono Q column. Then, the tagged hGH was thirdly purified by gel-filter chromatography. In the meantime, histidine-not-tagged human growth hormone (untagged hGH) was firstly purified by anion exchange chromatography using DEAE column and then secondly purified by anion exchange chromatography using Mono Q column. Then, the untagged hGH was thirdly purified by gel-filter chromatography.

As a method to confirm the biological activity of the purified recombinant protein, particularly human growth hormone, cell proliferation assay using mouse Nb2-11 cells displaying high growth-promoting activity is preferred, but not always limited thereto.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Construction of a Vector for the Expression of Human Growth Hormone

To construct a vector for the expression of human growth hormone, hGH gene was cloned.

Particularly, the present inventors requested BIONEER Co. (Daejeon, Korea) to synthesize the human growth hormone gene (NCBI Reference: NM_{—}000515.3: 141-719) for the expression of the recombinant human growth hormone with the insertion of NheI restriction enzyme cleavage site (GCTAGC) at 5′-end and XhoI restriction enzyme cleavage site (CTCGAG) at 3′-end (SEQ. ID. NO: 1). The synthesized cDNA was ligated to pET-28a (Novagen, Madison, Wis., USA) by using the restriction enzymes NheI and XhoI, leading to the construction of His-hGH expression vector expressing the recombinant human growth hormone that contains 6-histidine tag and thrombin cleavage site at N-terminal.

For the untagged hGH expression vector expressing the recombinant human growth hormone without histidine tag, cDNA containing NcoI restriction enzyme cleavage site (CCATGG) at 5′-end and XhoI restriction enzyme cleavage site (CTCGAG) at 3′-end was constructed by PCR using the above synthesized cDNA as a template with the forward primer 5′-ccatggcgatgttcccaaccatt-3′ (SEQ. ID. NO: 2) and the reverse primer 5′-ctcgagctagaagccacagct-3′ (SEQ. ID. NO: 3). The PCR product was digested with NcoI and XhoI and then ligated to NcoI and XhoI restriction enzyme sites of pET28a expression vector in order to express the untagged recombinant human growth hormone (untagged hGH). The nucleotide sequence of the gene for the expression was identified by automatic sequencing.

As a result, vectors for the expression of each 6-histidine-tag containing human growth hormone and 6-histidine-tag not-containing human growth hormone were constructed. The 6-histidine-tag containing human growth hormone was named His-hGH and the 6-histidine-tag not-containing human growth hormone was named untagged hGH.

### Example 2

### Investigation of the Expression and Extraction of Human Growth Hormone Using E. coli Expression System

In order to express the human growth hormone not-tagged with 6-histidine at N-terminal (untagged hGH) and the human growth hormone tagged with 6-histidine at N-terminal (His-hGH), E. coli BL21(DE3) was transformed with the vectors prepared above. The transformed E. coli BL21(DE3) cells were distributed in 500 of the fresh LB (Luria-Bretanu) medium containing 50 mg/ml kanamycin, 10 g/l. Bacto Tryptone, 5 g/l yeast extract, and 10 g/l NaCl by 10 ml, followed by culture at 37° C. until OD₆₀₀ reached approximately 0.6. The cultured E. coli culture solution was quick-frozen, which stood at 4° C. for 60 minutes. To induce the expression of human growth hormone in the E. coli culture solution above, 1 mM β-D-1-thiogalactopyranoside (IPTG) was added thereto. The E. coli cells were cultured at different temperatures (16° C., 20° C., 25° C., 30° C., and 37° C.). Then, the cultured E. coli cells (pallet) were collected. The recovered E. coli cells were lysed in 25 of lysis buffer (50 mM Tris-HCl containing 1 mg/ml lysozyme, 1× protease inhibitor cocktail (Roche, Spain), and 0.5 mM EDTA) by sonication, followed by centrifugation at 10,000×g for 20 minutes to obtain insoluble pellets and soluble fractions. The obtained pellets and fractions proceeded to gel electrophoresis (SDS-PAGE). The SDS-PAGE gel was stained with coomassie blue. For the quantitative analysis of human growth hormone, densitometry assay was performed with the soluble (S) and insoluble (P) proteins using ImageQuant™ TL 5.2 analysis software.

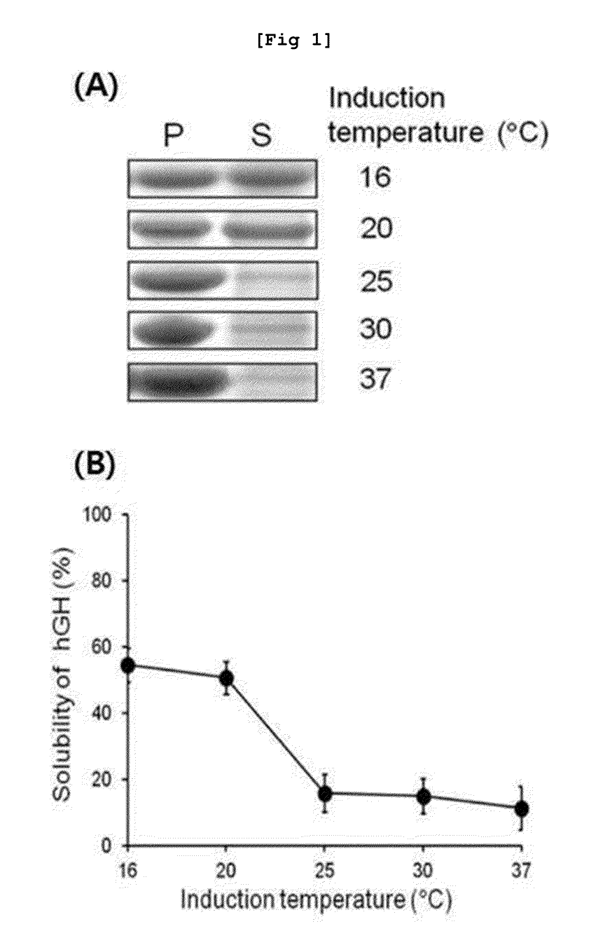

As a result, as shown in FIG. 1, the solubility of the extracted protein was improved when the expression was induced at 16˜20° C., compared with when the expression was induced at higher temperatures like (25˜37° C.) than the above (FIG. 1).

The protein expression was compared between when the hGH expressing E. coli cells were cultured at 37° C., indicating that the expression was induced at 37° C., and when the protein expression was induced at 16° C. which had been confirmed as the temperature being starting point of solubility increase, as shown in FIG. 1, by the same manner.

Particularly, E. coli cells in which the expression of human growth hormone was not induced (U); E. coli cells in which the expression of human growth hormone was induced at 37° C. and 16° C. by the treatment of IPTG (I); and E. coli cells treated with a lysis buffer after confirming the expression of human growth hormone therein (L); insoluble fraction obtained by centrifuging the E. coli cells treated with a lysis buffer after confirming the expression of human growth hormone therein (P); and soluble fraction obtained by centrifuging the E. coli cells treated with a lysis buffer after confirming the expression of human growth hormone therein (S) were all electrophoresed using 4˜12% SDS-PAGE gel, followed by staining with coomassie blue.

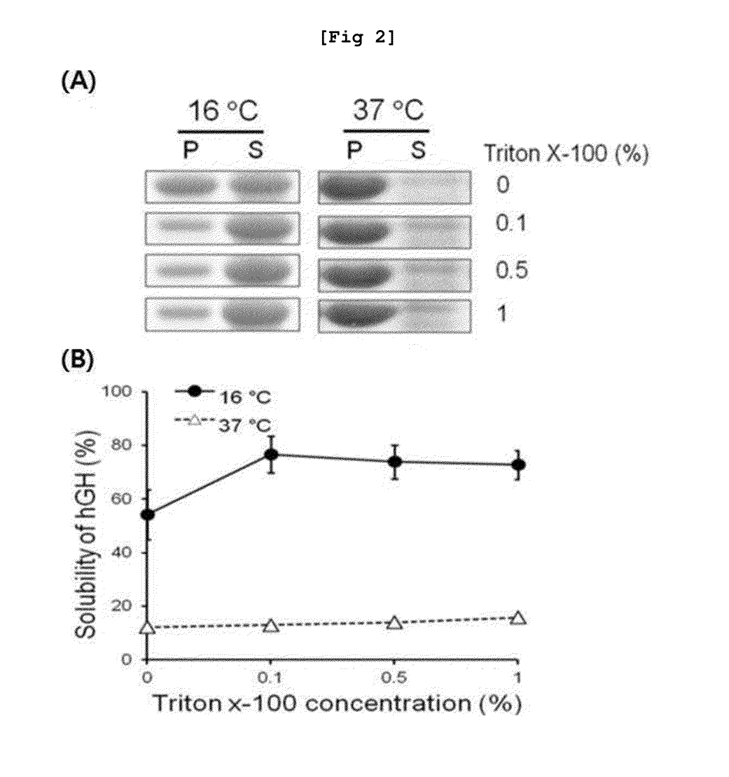

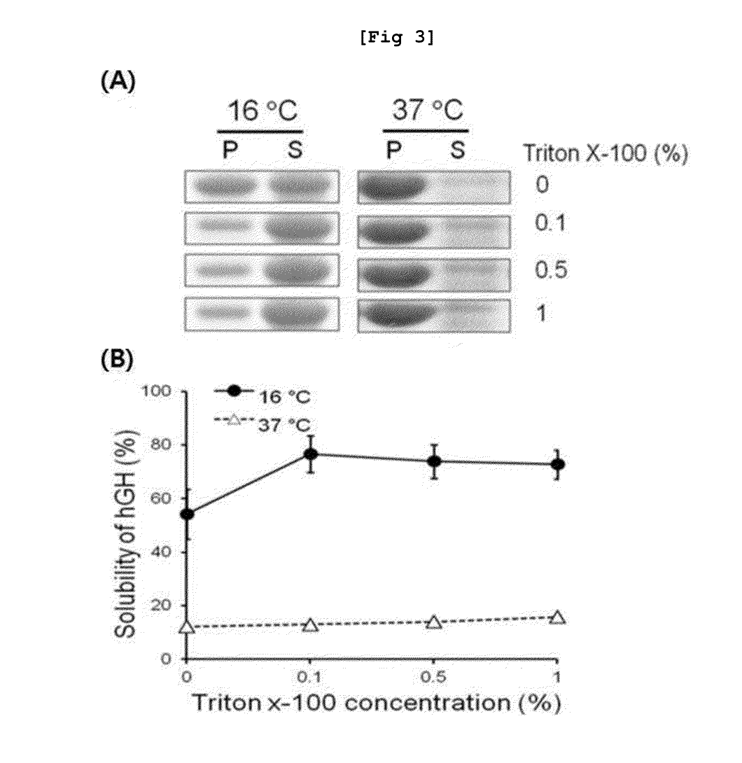

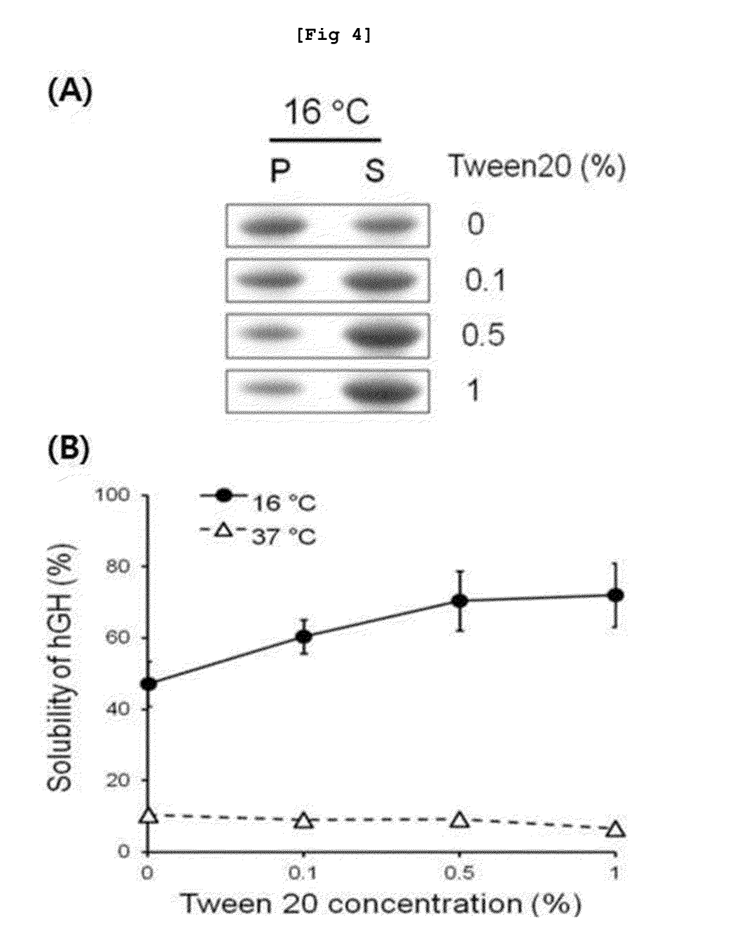

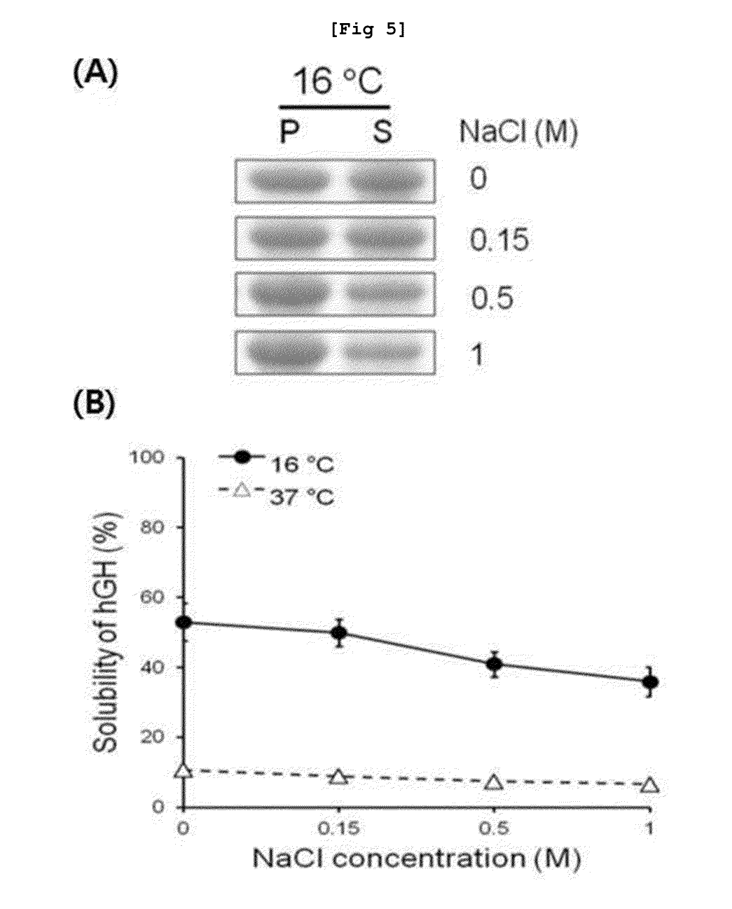

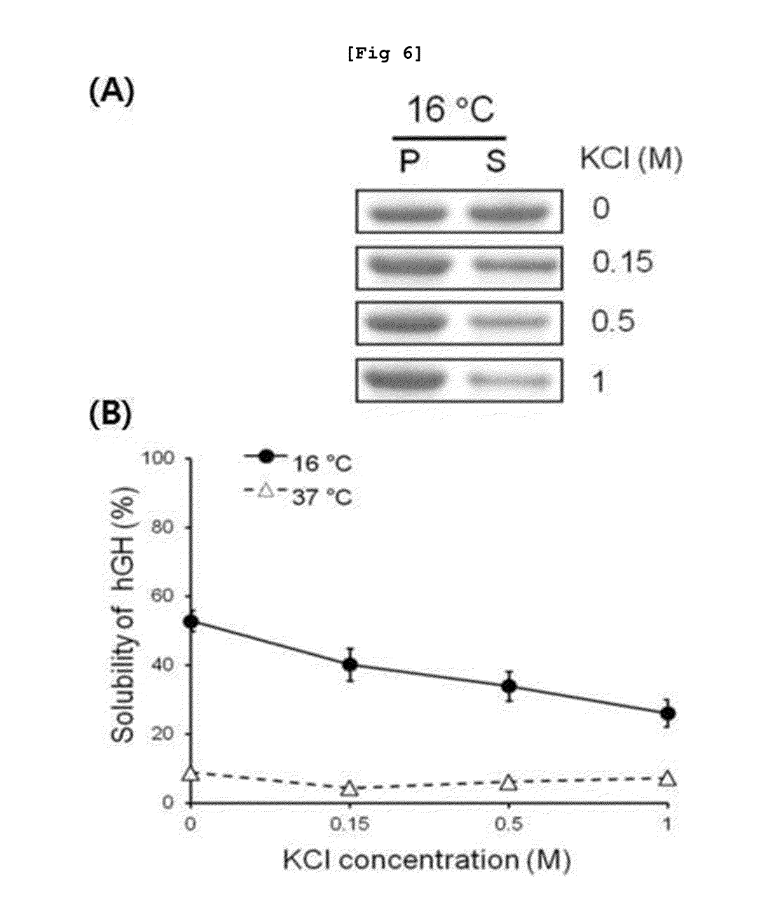

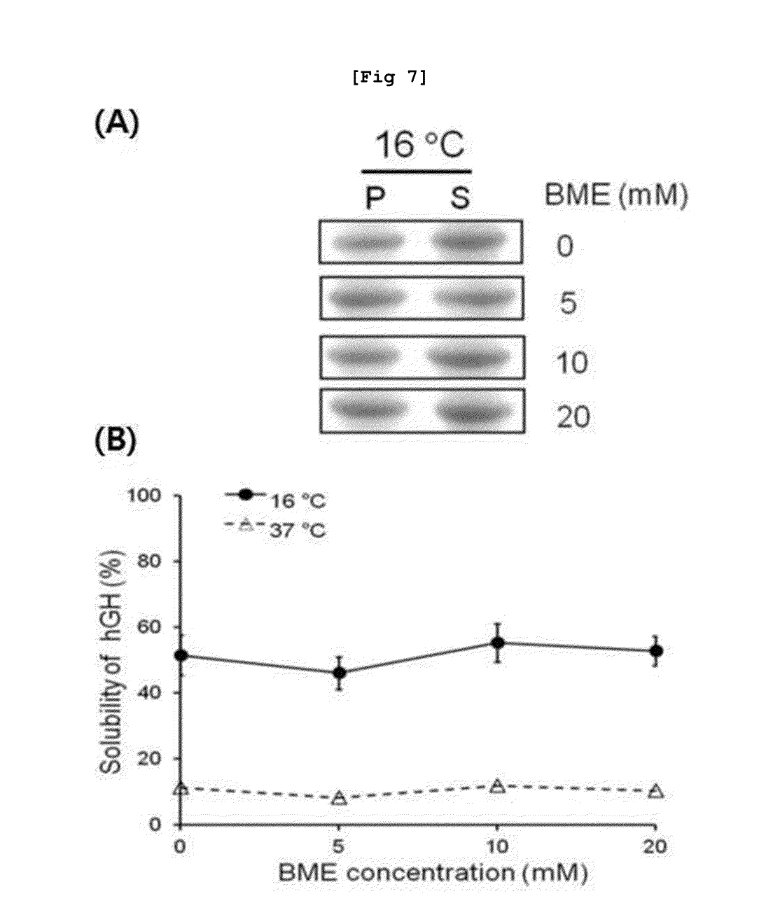

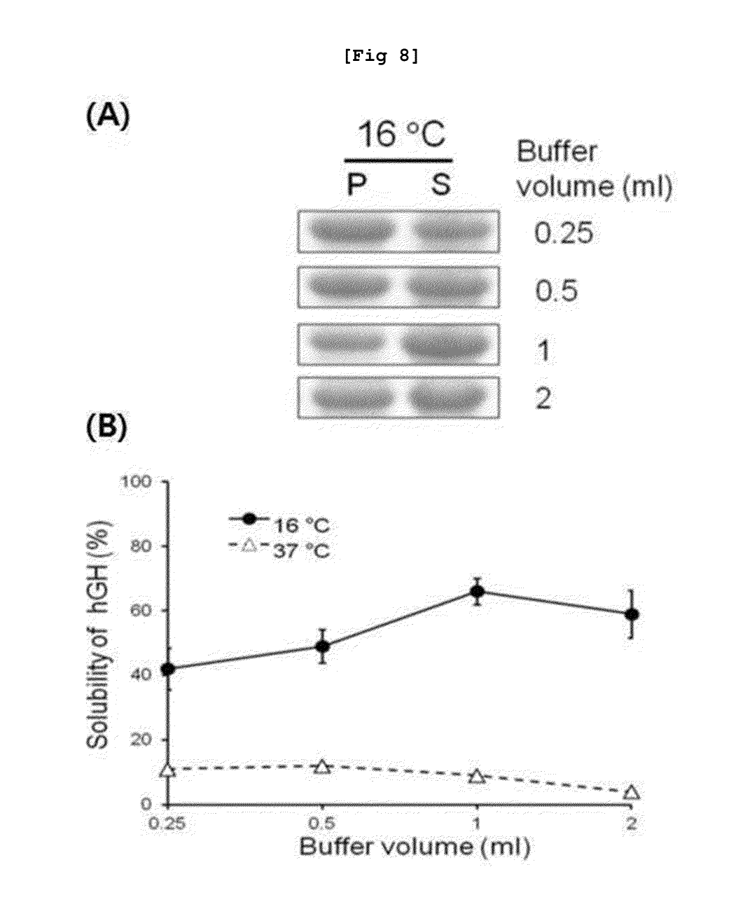

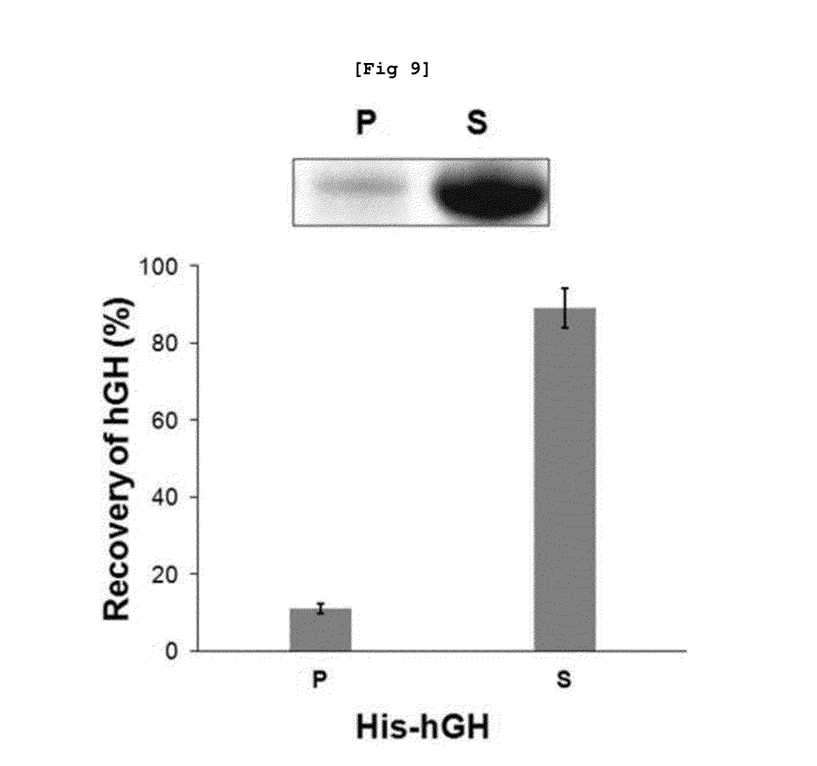

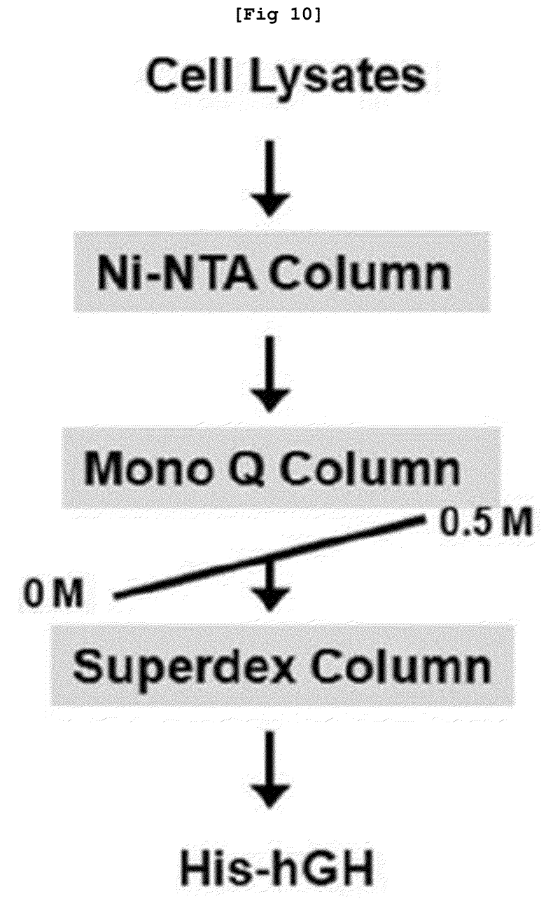

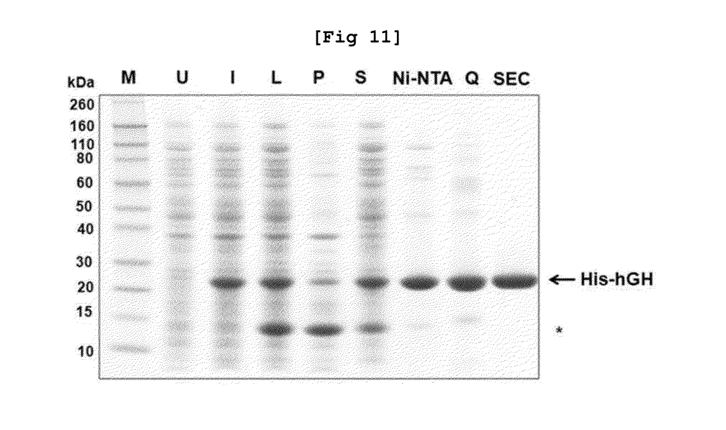

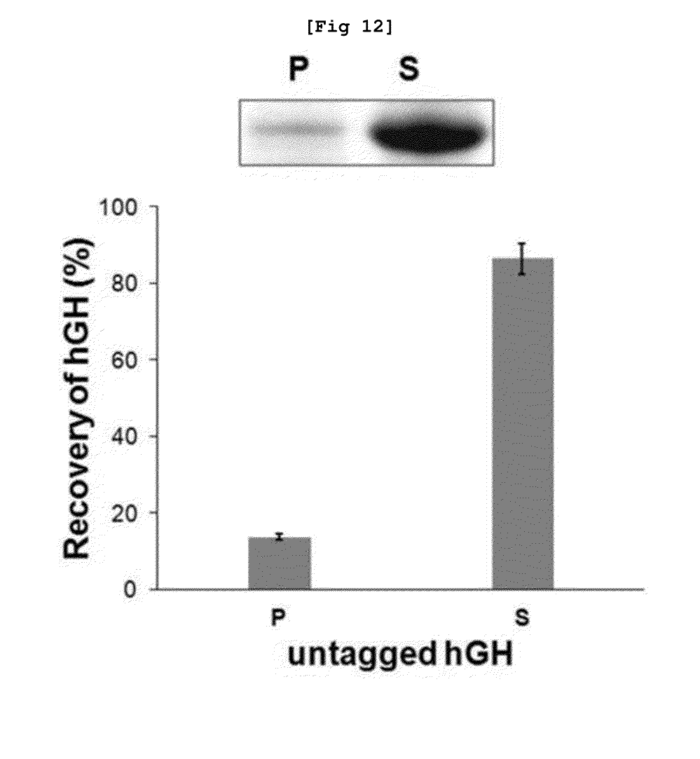

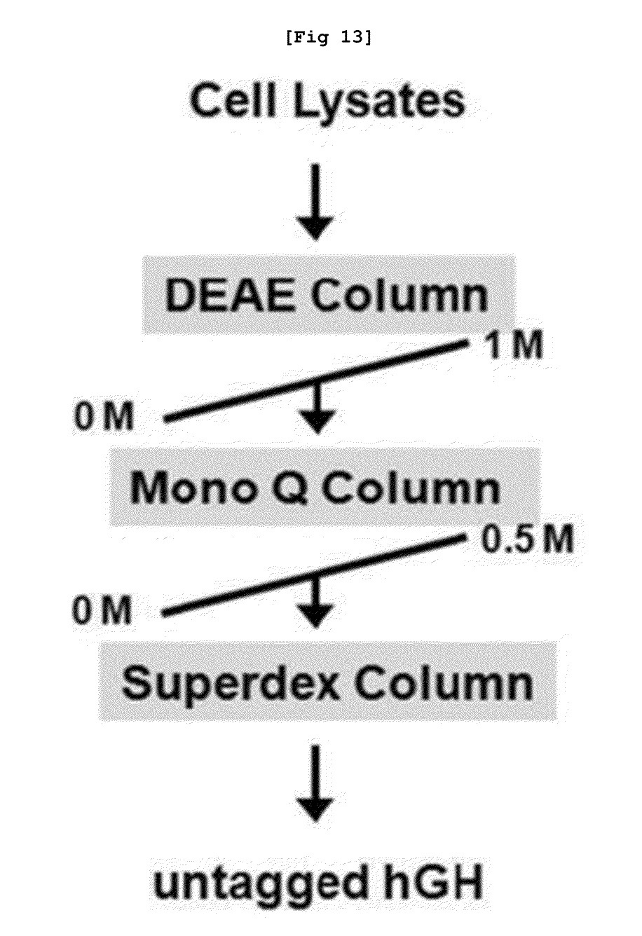

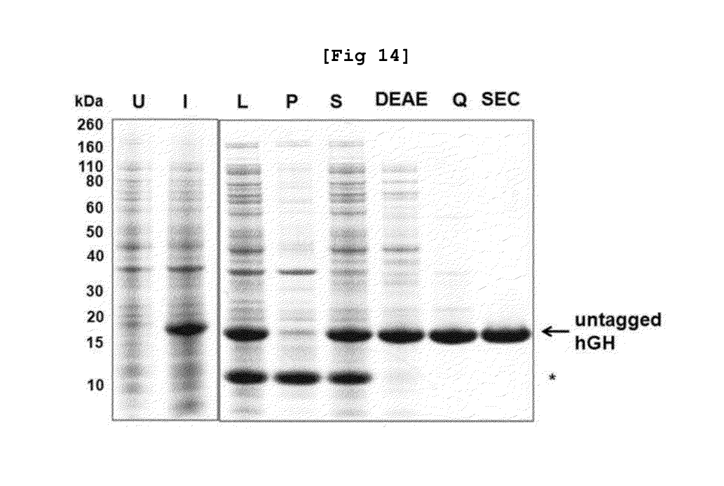

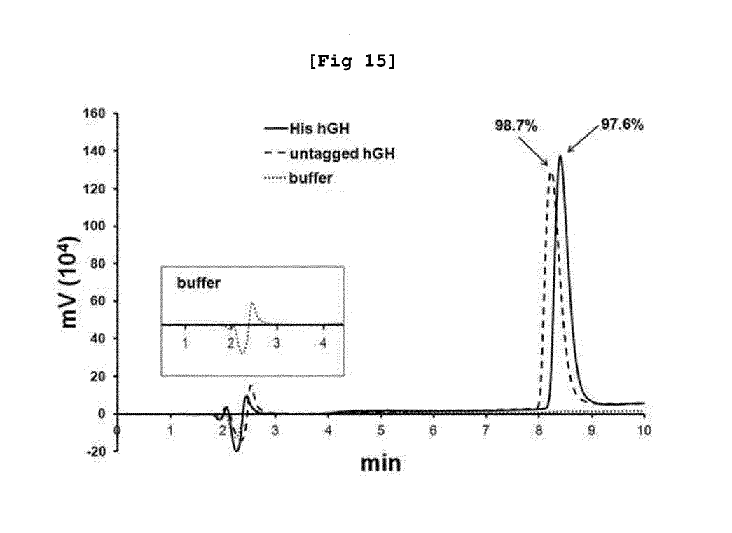

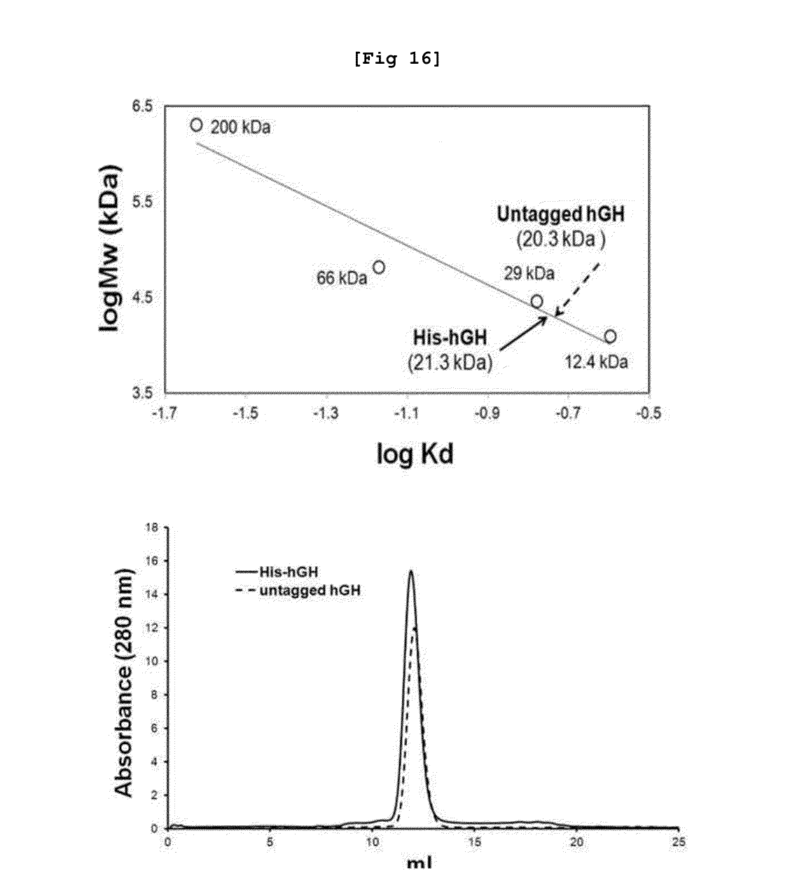

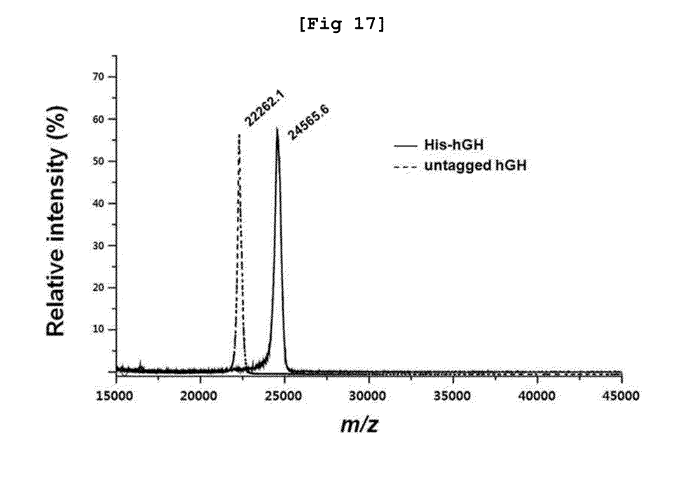

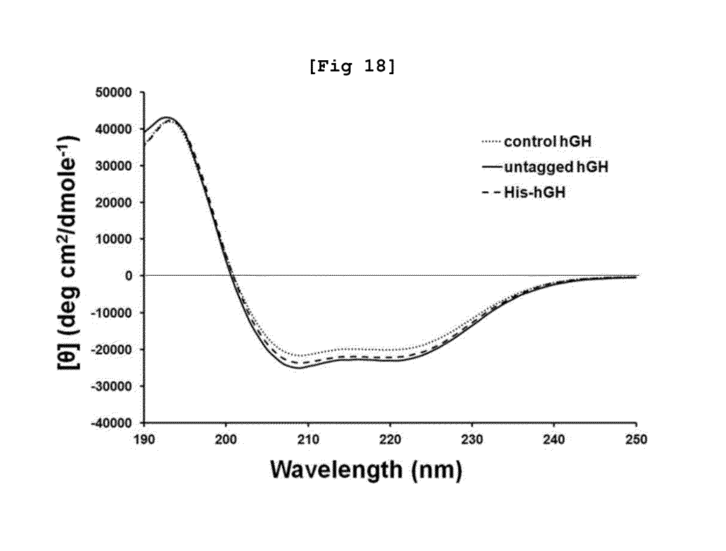

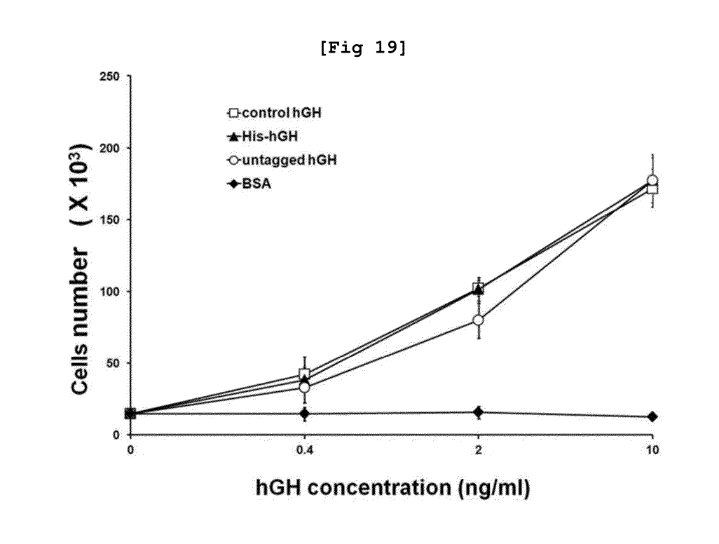

As a result, as shown in FIG. 2, the amount of the soluble fraction (S) obtained by centrifuging the E. coli cells treated with a lysis buffer after confirming the expression of human growth hormone induced at 16° C. was significantly increased compared with another soluble fraction obtained from the E. coli cells in which the expression of human growth hormone was induced at 37° C. (FIG. 2).

### Example 3

### Optimum Conditions of Lysis Buffer for the Solubilization of Insoluble Fraction

The present inventors investigated the optimum composition of lysis buffer in order to change insoluble fractions into soluble fractions by solubilizing the insoluble fractions.

Particularly, the optimum composition of lysis buffer for the insoluble fractions obtained by the same manner as described in Example 2 was confirmed by trying out the composition of lysis buffer as shown in Table 1.

TABLE 1
Composition of lysis buffer to obtain soluble
proteins from insoluble fractions

| Sample | Composition of Lysis Buffer | |
|---|---|---|
| Number | Additive (Conc.) | Composition |
| 1 | Triton X-100 | 50 mM Tris-HCl (pH 8.0) |
| | (0, 0.1, 0.5 or 1%) | containing 1 mg/ml, lysozyme, 1× |
| 2 | Tween 20 | protease inhibitor cocktail, and 0.5 |
| | (0, 0.1, 0.5 or 1%) | mM EDTA |
| 3 | NaCl | |
| | (0, 0.15, 0.5 or 1M) | |
| 4 | KCl | |
| | (0, 0.15, 0.5 or 1M) | |
| 5 | β-mercaptoethanol | |
| | (0, 50, 10 or 20 mM) | |

As a result, as shown in FIG. 3, the solubility was analyzed with the volume changes of Triton X-100, the non-ionic detergent, from 0.1 to 1%(v/v). It was confirmed that the lysis buffer supplemented with Triton X-100 was effective in dissolving human growth hormone (FIG. 3).

As shown in FIG. 4, the solubility was also analyzed with the volume changes of Tween 20, another non-ionic detergent, from 0.1 to 1%(v/v). As a result, it was confirmed that the addition of Tween 20 was as effective in dissolving human growth hormone as Triton X-100 (FIG. 4).

As shown in FIG. 5 and FIG. 6, the solubility was investigated over the changes of the concentrations of NaCl or KCl from 0.1˜1 M. As a result, it was confirmed that the lysis buffer supplemented with NaCl or KCl was not so effective in dissolving human growth hormone, and rather brought inhibiting effect on the solubility of human growth hormone (FIG. 5 and FIG. 6).

As shown in FIG. 7, the solubility was investigated over the volume changes of β-mercaptoethanol added to the lysis buffer. As a result, it was confirmed that the addition of β-mercaptoethanol did not change the solubility of human growth hormone at all (FIG. 7).

In addition, as shown in FIG. 8, the solubility was investigated over the amount of lysis buffer (50 mM Tris-HCl containing 1 mg/ml lysozyme, 1× protease inhibitor cocktail (Roche, Spain), and 0.5 mM EDTA) from 0.25˜2 ml used for 30 mg of the E. coli cells expressing human growth hormone. As a result, it was confirmed that the proper amount of lysis buffer for 30 mg of the pellet was 1 (FIG. 8).

Therefore, the optimum condition for the expression and extraction of human growth hormone using E. coli expression system was established via Example 1 and Example 3.

### Example 4

### Purification of Human Growth Hormone, the Recombinant Protein

The purification method after the extraction of human growth hormone was examined in this Example 4 based on the optimum conditions for the expression and extraction of human growth hormone established in Example 1˜Example 3. The E. coli BL21(DE3) cells expressing the recombinant human growth hormone (untagged hGH and His-hGH) were cultured in 250 of a culture medium, during which the expression of hGH was induced at 16° C. for 16 hours and then harvested (FIG. 9). The harvested cells were lysed in 25 lysis buffer (50 mM Tris-HCl (pH 8.0) containing 0.5 mM EDTA, 0.1% Triton X-100, 1 mg/ml lysozyme, and 1× protease inhibitor cocktail (Roche, Spain)) by sonication, followed by centrifugation at 10,000 g for 20 minutes. Then, the obtained pellet was purified as shown in FIG. 10.

<4-1> Purification of his-hGH

### <4-1-1> Affinity Chromatography (Using Ni-NTA Column)

After the centrifugation, the supernatant of His-hGH dissolved as optimized was loaded in the column filled with Ni-NTA agarose (1 ml) (Qiagen, Valencia, Calif.) beads. His-hGH protein injected in the column was washed with washing buffer (Table 2) in three times the volume of the column and then eluted using 10 elution buffer (Table 2), leading to the primary purification of His-hGH. The fractions containing His-hGH were dialyzed in anion exchange column buffer (50 mM Tris-HCl (pH 8.0) and 10% glycerol).

### <4-1-2> Anion-Exchange Chromatography (Using Mono Q Column)

The dialyzed fraction was eluted using anion-exchange chromatography 5/50 Mono Q column (GE Healthcare, USA) according to the linear density gradient of NaCl (0˜500 mM), leading to the secondary purification.

### <4-1-3> Gel-Filter Chromatography (Using Superdex 200 Column)

Lastly, the fraction containing His-hGH was thirdly purified by gel filtration using HiLoad 26/30 Superdex 200 column and gel filtration column buffer (50 mM Tris-HCl (pH 8.0) containing 150 mM NaCl and 10% glycerol) (FIGS. 9˜11).

The purified protein was dialyzed in storage buffer (10 mM Na₂HPO₄, pH 7.4, 0.5% glycine, 2.25% mannitol) and stored. The concentration of the protein was measured by Bradford assay and Bicinchoninic acid (BCA) protein assay using bovine serum albumin (BSA) as the standard. The purity was measured by SDS-PAGE and silver staining with comparing the theoretical molecular weight (˜21 kDa) (Table 3).

TABLE 2
Buffer composition for affinity chromatography

| Composition | Washing Buffer* | Elution Buffer* |
|---|---|---|
| NaCl | 150 mM | 150 mM |
| Imidazole | 10 mM | 200 mM |
| Triton X-100 | 0.1% | 0.1% |
| Glycerol | 10% | 10% |

*1× Phosphate buffered saline (PBS, pH 7.4) comprising the above composition was used as washing and elution buffer.

As a result, as shown in Table 3 and FIGS. 9˜11, His-hGH insoluble pellet and soluble fractions were obtained from the primarily extracted hGH whose expression was induced at 16° C. for 16 hours in E. coli under the optimized condition (FIG. 9), which proceeded to affinity chromatography using Ni-NTA column, anion-exchange chromatography using Mono Q column, and gel-filter chromatography using superdex 200 column, leading to the efficient purification of His-hGH (FIG. 10 and FIG. 11). The purity of the protein after this purification was 97.9% (Table 3).

TABLE 3
His-hGH separated/purified from E. coli

| | Total | | | |
|---|---|---|---|---|
| | Protein^{a} | hGH Purity^{b} | hGH^{c} | Total Yield |
| Purification Step | (mg) | (%) | (mg) | (%) |
| Soluble fraction | 65.4 | 35.5 | 23.2 | 100.0 |
| Ni-NTA column | 29.9 | 70.0 | 21.0 | 90.2 |
| Mono Q column | 15.6 | 93.9 | 14.7 | 63.2 |
| Superdex 200 column | 10.2 | 97.9 | 10.0 | 42.9 |

^{a}The total protein was obtained from 250 ml culture medium.
^{b}The hGH purity was determined by densitometry using coomassie blue stained gel.
^{c}Relative ratio to the total protein was calculated.

### <4-2> Purification of Untagged hGH

### <4-2-1> Anion-Exchange Chromatography (Using DEAE Column)

After the centrifugation, the supernatant of untagged hGH (hGH not-tagged with His) dissolved as optimized was dialyzed in anion exchange column buffer (50 mM Tris-HCl (pH 8.0) and 20% glycerol). The dialyzed fraction was eluted by anion-exchange chromatography using DEAE column (1 ml) (GE Healthcare, USA) according to the linear density gradient of NaCl (0˜1 M), leading to the primary purification.

### <4-2-2> Anion-Exchange Chromatography (Using Mono Q Column)

The secondary purification was performed by anion-exchange chromatography using 5/50 Mono Q column (GE Healthcare, USA) by the same manner and condition as described above for the primary purification of His hGH.

### <4-2˜3> Gel-Filter Chromatography (Using Superdex 200 Column)

The third purification was performed by gel-filter chromatography by the same manner and condition as described above for the primary purification of His-hGH.

The purified protein was dialyzed in storage buffer (10 mM Na₂HPO₄, pH 7.4, 0.5% glycine, 2.25% mannitol) and stored thereafter. The concentration of the protein was measured by Bradford assay and Bicinchoninic acid (BCA) protein assay using BSA as the standard. The purity was measured by SDS-PAGE and silver staining with comparing the theoretical molecular weight (˜21 kDa).

As a result, as shown in Table 4 and FIGS. 12˜14, untagged-hGH insoluble pellet and soluble fractions were obtained from the primarily extracted hGH whose expression was induced at 16° C. for 16 hours in E. coli under the optimized condition (FIG. 12), which proceeded to anion-exchange chromatography using DEAE column and Mono Q column, and gel-filter chromatography using superdex 200 column, leading to the efficient purification of untagged-hGH (FIG. 13 and FIG. 14). The purity of the protein after this purification was 97.2% (Table 4).

TABLE 4
Untagged-hGH separated/purified from E. coli

| | Total | | | |
|---|---|---|---|---|
| | Protein^{a} | hGH Purity^{b} | hGH^{c} | Total Yield |
| Purification Step | (mg) | (%) | (mg) | (%) |
| Soluble fraction | 56.2 | 37.2 | 20.9 | 100.0 |
| Ni-NTA column | 31.6 | 55.6 | 17.6 | 84.0 |
| Mono Q column | 12.4 | 93.7 | 11.6 | 55.5 |
| Superdex 200 column | 8.7 | 97.2 | 8.5 | 40.5 |

^{a}The total protein was obtained from 250 ml culture medium.
^{b}The hGH purity was determined by densitometry using coomassie blue stained gel.
^{c}Relative ratio to the total protein was calculated.

### Example 5

### Characteristics of the Purified Protein

### <5˜1> Purity of the Purified Protein

To measure the purity of the protein, the purified protein obtained in Example 4 was analyzed by reverse-phase high-performance liquid chromatography (PR-HPLC).

Particularly, to measure the purity of the protein obtained in Example 4, RP-HPLC equipped with Kinetex C18 column (2.6 μm, 150×2.10 mm; Phenomenex, Torrance, Calif., USA) was performed. Buffer A (0.1% trifluoroacetic acid (TFA) in H₂O) and buffer B (0.1% trifluoroacetic acid (TFA) in acetonitrile (ACN)) were used. The linear gradient of buffer B was 28%˜100%, and elution was performed at 40° C. The flow rate was 0.2 ml/min, and OD₂₂₀ was measured.

As a result, as shown in FIG. 15, the purity of the untagged-hGH was 98.7% and the purity of His-hGH was 97.6% (FIG. 15).

### <5-2> Size of the Purified Protein

To measure the size of the protein, the purified protein obtained in Example 4 was analyzed by Analytical size exclusion chromatography (SEC).

Particularly, to confirm the size of the protein obtained in Example 4, the protein was loaded in RP-HPLC device equipped with superdex 75 10/300 GL column (GE Healthcare, USA). As the moving phase, Tris-HCl buffer (pH 8.0) comprising 150 mM NaCl and 10% glycerol was used. The flow rate was 0.5 ml/min. OD₂₈₀ was measured to confirmed the protein size. As the standard markers, 200 kDa blue dextran, 66 kDa BSA, 29 kDa carbonic anhydrase, and 12.4 kDa ribonuclease A were used. These were displayed by the log size, followed by comparison.

As a result, as shown in FIG. 16, the mass of His-hGH was 21,314 Da and the mass of untagged hGH was 20,312 Da (FIG. 16).

### <5-3> Molecular Weight of the Purified Protein

To confirm the molecular weight of the protein, the purified protein obtained in Example 4 was analyzed by Matrix-assisted Laser Desorption Ionization-Time of Flight of Flight Mass Spectrometry (MADI-TOF).

Particularly, 1 mg/ml of the protein obtained in Example 4 was mixed with the MALDI matrix α-cyano-4-hydroxycinnamic acid at the ratio of 1:10 (v/v), followed by spotting thereof on the MALDI mass spectrometry plate, which was then analyzed with Autoflex III Smartbeam (Bruker Daltonics, USA). At this time, peptides and protein correction kit (Sigma, USA) were used. Mass spectrometry was performed in the cation mode in the range of 15,000˜45,000 m/z, leading to the confirmation of the mass of His-hGH and untagged hGH.

As a result, as shown in FIG. 17, the mass of His-hGH was 22,262 Da, and the mass of untagged-hGH was 24,565 Da (FIG. 17).

### <5-3> Secondary Structure of the Purified Protein

To confirm the secondary structure of the protein, the purified protein obtained in Example 4 was analyzed by circular dichroism (CD).

Particularly, the protein obtained in Example 4 was loaded in a quartz cuvette (path length: 0.1 mm), followed by analyzing with J-815 circular dichroism spectropolarimeter (Jasko, Japan) (wavelength: 200˜250 nm, band width: 0.1 nm, scan speed: 50 nm/min, reaction speed: 10 seconds). As the control, the commercial hGH (LG Life Sciences, Korea) was purchased and the secondary structure thereof was confirmed by the same manner as described above.

As a result, as shown in FIG. 18, it was confirmed that His-hGH and untagged hGH had α-helix structure, which is the same secondary structure as the one of the commercial hGH (FIG. 18).

### Example 6

### Recombinant Human Growth Hormone Activity

To confirm the activity of the recombinant human growth hormone, MTS assay was performed based on the fact that 3-(4,5-dimethylthiazol-2-yl)-2,5-diphenyl tetrazolium bromide (MTS) is reduced into MTS-formazan mediated by the generation of NADPH or NADH caused by dehydrogenase in living mitochondria (J. Immunol Methods 65; 1983, 55-63).

Particularly, NB2-11 cells, the mouse originated T-lymphoma cell line displaying prolactin (PRL) dependence, were inoculated in RPMI 1640 supplemented with 10% FBS (Gibco/Invitrogen, USA), 10% horse serum (HS; Gibco/Invitrogen, USA), and 1% penicillin-streptomycin, followed by culture in a 37° C. 5% CO₂ incubator for 48 hours. Upon completion of the culture, the NB21-11 cells were washed with a FBS-free medium, which were then distributed in a 96-well plate (20,000 cells/well). His-hGH or untagged hGH obtained in Example 4 was treated to each well of the plate at different concentrations of 0.4, 2, and 10 ng/ml, followed by culture in a 37° C. 5% CO₂ incubator for 48 hours. 20 μl of MTS reagent was added to each well of the plate, followed by further culture for 2 hours. OD₄₉₀ was measured with a microplate reader (BioLad, USA) to confirm cell proliferation. BSA was used as the negative control, and the commercial hGH (LG Life Sciences, Korea) was used as the positive control. The cell proliferation in the controls was also confirmed by the same manner as described above.

As a result, as shown in FIG. 19, compared with the negative control, the recombinant human growth hormones His-hGH and untagged-hGH were confirmed to have efficient cell proliferation activity (FIG. 19).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for producing a soluble target protein comprising the following steps:
(1) performing primary culture of E. coli transfected with an expression vector encoding a target protein;
(2) quick-freezing the E. coli primary culture fluid at 0˜10° C., which stands as it is for 30˜180 minutes;
(3) adding an inducer to the E. coli culture fluid to induce the expression of the target protein; and
(4) culturing the E. coli culture fluid added with the said inducer at 15˜25° C. for 8˜18 hours.

2. The method for producing a soluble target protein according to claim 1, wherein the target protein of step (1) is human growth hormone.

3. The method for producing a soluble target protein according to claim 1, wherein the inducer of step (3) is 0.1˜1 mM β-D-1-thiogalactopyranoside (IPTG).

4. A method for extracting and purifying a soluble target protein comprising the following steps:
(1) performing primary culture of E. coli transfected with an expression vector encoding a target protein;
(2) quick-freezing the E. coli primary culture fluid at 0˜10° C., which stands as it is for 30˜180 minutes;
(3) adding an inducer to the E. coli culture fluid to induce the expression of the target protein;
(4) culturing the E. coli culture fluid added with the said inducer at 15˜25° C. for 8˜18 hours.
(5) lysing the E. coli cells of step (4) by using a lysis buffer;
(6) sonicating the E. coli cells, which are then centrifuged to recover the soluble target protein; and
(7) purifying the soluble target protein.

5. The method for extracting and purifying a soluble target protein according to claim 4, wherein the soluble target protein in step (7) is characterized by being not inhibited in its biological activity.

6. The method for extracting and purifying a soluble target protein according to claim 4, wherein the lysis buffer of step (5) contains a non-ionic detergent.

7. The method for extracting and purifying a soluble target protein according to claim 4, wherein the non-ionic detergent is 0.01˜2% (v/v) Triton X-100 or Tween 20.

8. The method for extracting and purifying a soluble target protein according to claim 4, wherein the lysis buffer of step (5) does not contain any salt.

9. The method for extracting and purifying a soluble target protein according to claim 8, wherein the salt is NaCl or KCl.

10. The method for extracting and purifying a soluble target protein according to claim 4, wherein the amount of the used lysis buffer is 0.25˜2 ml for 30 mg of E. coli cell pellet.

11. The method for extracting and purifying a soluble target protein according to claim 4, wherein the soluble target protein of step (7) is histidine (His) tagged human growth hormone or histidine not-tagged human growth hormone.

12. The method for extracting and purifying a soluble target protein according to claim 11, wherein the histidine tagged human growth hormone is purified by one or more methods selected from the group consisting of affinity chromatography, anion exchange chromatography, and gel-filter chromatography.

13. The method for extracting and purifying a soluble target protein according to claim 11, wherein the histidine not-tagged human growth hormone is purified by anion exchange chromatography, gel-filter chromatography, or both of anion exchange chromatography and gel-filter chromatography.

14. The method for extracting and purifying a soluble target protein according to claim 12, wherein the affinity chromatography column is Ni-NTA column.

15. The method for extracting and purifying a soluble target protein according to claim 12, wherein the anion exchange chromatography column is Mono Q column.

16. The method for extracting and purifying a soluble target protein according to claim 13, wherein either DEAE column or Mono Q column is used for the anion exchange chromatography or both of DEAE column and Mono Q column are used stepwise for the anion exchange chromatography.

17. The method for extracting and purifying a soluble target protein according to claim 12 or claim 13, wherein the gel-filter chromatography column is superdex 200 column.


SEQUENCE LISTING (3 sequences)

SEQ ID NO: 1 - DNA, 591 bp, Artificial Sequence
  Nhel-cDNA-Xhol
gctagcatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctcgag

SEQ ID NO: 2 - DNA, 23 bp, Artificial Sequence
  untagged hGH_F
ccatggcgatgttcccaaccatt

SEQ ID NO: 3 - DNA, 21 bp, Artificial Sequence
  untagged hGH_R
ctcgagctagaagccacagct